(19) United States
(12) Patent Application Publication
(10) Pub. No.: US 2022/0325314 A1
(43) Pub. Date: Oct. 13, 2022

(54) DETECTOR FOR MONITORING MICROBIOLOGICAL CONTAMINATION IN LIQUID STORAGE

(71) Applicant: TRITON SYSTEMS, INC., Chelmsford, MA (US)

(72) Inventor: Baris UNAL, Lowell, MA (US)

(21) Appl. No.: 17/584,779

(22) Filed: Jan. 26, 2022

Related U.S. Application Data
(60) Provisional application No. 63/141,776, filed on Jan. 26, 2021.

(51) Int. Cl. C12Q 1/04; G01N 27/02; G01N 33/28

(52) U.S. Cl. CPC C12Q 1/04; G01N 27/028; G01N 33/2835

(57) ABSTRACT

Described herein is a system for detecting microbial contamination in a liquid storage container, such as a fuel storage tank or a water storage tank. The system can include one or more impedance sensor arrays positioned within the fuel storage container and a computer system coupled to the impedance sensor arrays. The computer system configured to detect microbial growth within the fuel storage container based on changes in impedance across the impedance sensor array. The system can be embodied as a standalone system or can be incorporated into the fuel tank's existing systems.

### CROSS-REFERENCE TO RELATED APPLICATIONS

This application claims priority to U.S. Provisional Application Ser. No. 63/141,776 filed on Jan. 26, 2021 titled Detector for Monitoring Microbiological Contamination in Liquid Storage, the contents of which are incorporated herein by reference in its entirety.

### GOVERNMENT LICENSE RIGHTS

This invention was made with government support under contract number FA8100-19-P-0004/2601 awarded by the U.S. Air Force. The government has certain rights in the invention.

### BACKGROUND

Microbial contamination and the resulting biodeterioration of stored fuel are a significant and pervasive problem that adversely impacts a large sector of the fuel distribution infrastructure within both civilian and military sectors. Although microbial organisms are typically inactive in pure fuel (i.e., fuel in which the water activity (aw) is <0.9), the ubiquitous presence of water in fuel systems, coupled with an overall favorable growth environment within storage tanks, leads to inevitable accumulation of potentially biodeteriogenic biomass. Fuel systems have been observed to support a large diversity of different microorganisms, including bacteria and fungi (yeasts and molds), which will grow and propagate into biofilms. These are dense, robust, persistent networks of microbial organisms embedded in a complex matrix of excreted extracellular polymeric substance (EPS). A single biofilm can consist of single microbial species or multiple types of coexisting organisms.

Within a contaminated fuel system, the density of organisms and the biochemical activity is significantly greater within the biofilm than the surrounding fluid phases. The physical and chemical properties of the biofilm are therefore also substantially different than that of the fluids. Biomass is continually sloughing off from mature biofilms as “flocs” which can be distributed elsewhere in the fuel system, and nucleate new biofilm formation on pristine surfaces. The accumulated biomaterial can have detrimental effects on the downstream components, by clogging lines and filters, fouling tank gauges, and leading to microbiologically influenced corrosion (MIC). In long-term storage systems, extensive accumulation of microbes can result in metabolic consumption of aliphatic hydrocarbons and additives in the fuel itself. These detrimental effects are most likely to manifest in low-turnover systems where large quantities of biomass have time to accumulate.

Tank cleaning maintenance actions cost an estimated $15,000 per tank for ground fuels. Microbially contaminated fuel is generally required to be disposed of due to its unsuitability for use, costing an estimated $5.00 per gallon for disposal, not including the cost of replacement fuel. Traditional tank cleaning methods have historically incompletely sterilized a tank after cleaning, and as a result the remaining microbial biofilms will generally re-contaminate the tank within 12-18 months. Fuel system condition monitoring is a process used to determine if and when actionable steps need to be taken to prevent detrimental effects arising from biological contamination (please see attached file for more details). The central aspects of this process, sample collection, testing, and data analysis, seem simple enough, but the practicality of proper condition monitoring is impeded by several challenges resulting from inherent aspects of the fuel systems and the contamination itself. The conventional diagnostics methods for evaluating microbial contamination in stored fuel involves removing aliquots of the liquids and performing traditional biochemical-based analysis. There are practical limitations with the specific analytical methods currently utilized. Biochemical assays, such as cytoplasmic nucleotide adenosine triphosphate (cATP) assays, can be performed on-site and provide quantitative detection of the total microbial burden but lack specificity. On the other hand, culture testing can in principle offer more specificity and capability to quantify low levels of microbes, but it requires a laboratory environment, requires several days to obtain results, and at any given state of growth conditions, is unlikely to detect more than 1% of the total microbial population present. Quantitative PCR can readily identify microbes with extremely high specificity and sensitivity, but also requires a laboratory environment and can be labor intensive. In all cases, each sampling and analysis event only provides data for a single time point from a specific volumetric aliquot.

Regardless of the specific method employed, a small volume of sample must be removed from the tank for analysis. Yet, the biomass of interest is heterogeneously distributed throughout the storage tank, and has a propensity to grow on surfaces and at interfaces, rather than in the bulk volume, which makes acquiring a representative sample from a single or few locations challenging. For example, significant variation is found to be correlated to the depth within the fuel from which the sample was acquired. Sample acquisition is also constrained by the locations of access ports on the tank and may prevent adequate microbial sampling, whether the microbial biomass is located in the water layer, including the water-fuel interface, or the tank wall or other surfaces. The sampling technique often entails simply lowering an open container, essentially a bucket, to the bottom of the fuel tank, and then pulling the container back up, whereby it collects a portion of the water bottom, the interface, and the fuel phases. Such a crude approach has obvious shortcomings, in that the samples acquired are coming from single location in the tank, i.e. the column of fuel and water directly below the access port, and does not acquire any samples near the surfaces of the tank or other components in the fuel tank, such as the automated tank gauge (ATG). Regardless of the specific method, the requirement for removing a sample decouples the sampling from the measurement.

The overall tedious procedure of collecting and processing the sample must be performed repetitively to produce a time-based trend analysis, which will enable more informative condition monitoring. Given that the same general analytical techniques used for monitoring are also employed to scientifically study biocontamination, the inherent limitations of these research tools have led to an incomplete understanding of the problem. A more practical and informative approach would therefore entail a platform that performs measurements automatically, continuously, and can perform in situ measurements inside the storage tank.

Accordingly, the embodiments of sensing systems described herein provide comprehensive, sensitive, and robust monitoring of microbial activity within fuel storage and distribution systems. The sensing systems can be embodied as standalone systems or incorporated into existing ATG platforms or other fuel tank components. The resulting ability to continuously monitor for microbial activity within multiple locations of a fuel system provides an unprecedented degree of diagnostic data collection for detecting the presence of potentially problematic contamination earlier than what is currently possible. Being able to comprehensively and precisely monitor microbial growth with a fuel system is absolutely critical because improper monitoring and insufficient preventative action can lead to accumulation of significant biomass in a fuel tank, which can result in the need to drain the fuel and completely clean the tank. As mentioned above, this is a costly procedure. Detection of microbial activity at its earliest stages and closely observing its growth behavior can provide the fuel tank operator with actionable information to initiate a lower cost measure, such as adding biocides. This earlier prevention can in turn reduce the likelihood of needing a costly tank sterilization and can also help mitigate the spreading to other vehicles or fuel system components.

### SUMMARY

Described herein is a diagnostic platform for direct measurement of microbiological contamination within a fuel system. The platform is based upon a unique design consisting of impedance microsensors capable of detecting microbes within a fuel tank and provides notification when contamination is first identified, so that early preventative steps can be taken to prevent spreading of the contamination. The described diagnostic platform is highly versatile and directly adaptable to a variety of fuel storage and distribution systems, including such systems used by the U.S. Air Force, other DOD agencies, and the commercial sector. It is likewise compatible with all aviation turbine and diesel fuel grades used by the U.S. Air Force.

In addition to providing the benefits of being automated and continuous, the microsensors described herein are capable of directly measuring presence and propagation of the biofilm in situ and in real time. This approach couples the comprehensive sampling and the sensing aspects together, enabling both improved sensitivity and more reliable readings. This is a significant capability improvement over conventional methods, and in addition to closing the capability gap for routine diagnostics, the new technology ultimately offers a completely new analytical approach to improve fundamental understanding of biofilm formation and accumulation within a fuel system to enable pro-active measures.

In some embodiments, the present disclosure is directed to a system for detecting microbial contamination in a fuel storage container, the system comprising: an impedance sensor array positioned within the fuel storage container, the impedance sensor array configured to measure an impedance across the impedance sensor array; and a computer system coupled to the impedance sensor array, the computer system configured to: receive the impedance from the impedance sensor array, compare the impedance to a threshold impedance, and determine whether microbial growth is present within a vicinity of the impedance sensor array based on the comparison.

In some embodiments, the computer system is used to further quantify an amount of the microbial growth based on the comparison.

In some embodiments, the computer system is further configured to output a user alert based on the comparison.

In some embodiments, the threshold impedance has been previously characterized for the fuel tank by the computer system.

### FIGURES

The accompanying drawings, which are incorporated in and form a part of the specification, illustrate the embodiments of the invention and together with the written description serve to explain the principles, characteristics, and features of the invention. In the drawings:

FIG. 1 depicts a diagram of a fuel tank including a microbial biosensor system, in accordance with an embodiment of the present disclosure.

FIG. 2 depicts a sectional view of a fuel tank including a microbial biosensor system integrated into the fuel tank's automatic tank gauge (ATG) system, in accordance with an embodiment of the present disclosure.

FIG. 3 depicts a diagram of a sensor module for a microbial biosensor system, in accordance with an embodiment of the present disclosure.

### DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In some embodiments, the present disclosure is directed to a microsensor array assembly that is configured to detect the presence and accumulation of contaminants, such as microbes. The microsensor array assembly can be used in combination with liquid tanks, such as fuel tanks or water tanks, for detecting contamination within the tank, for example. In some embodiments, the present disclosure is directed to a microsensor array assembly in combination with the fuel tank. Although this disclosure is written in the context of fuel storage tanks, the devices, apparatus, and methods described herein are useful in other storage tanks. Significantly, we believe, but aren't bound by this theory, that contamination occurs in the water phase in fuel storage tanks, but appears to be present in the fuel phase due to contamination during sampling.

### Microbial Biosensor System for Fuel Storage

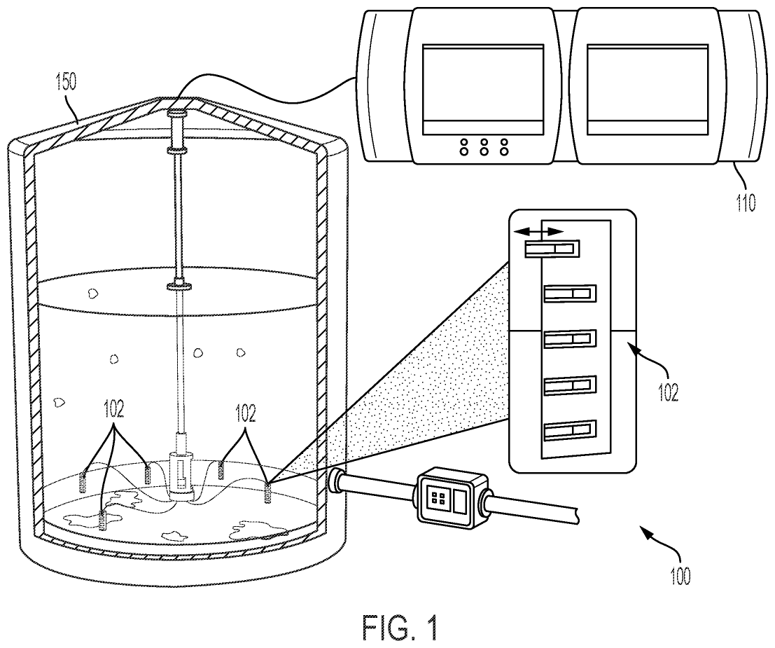

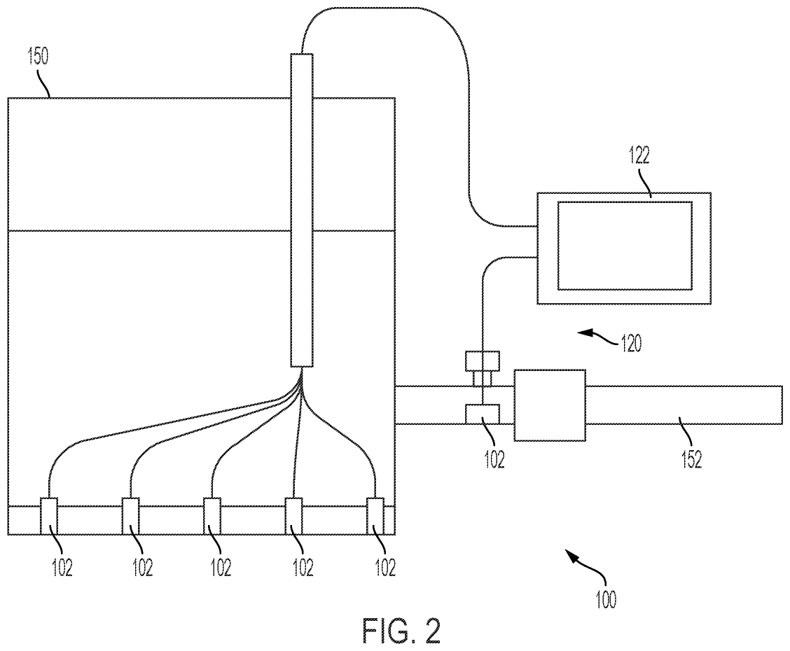

The microbial biosensor system 100 can include one or more impedance sensor arrays 102. The impedance sensor arrays 102 can be used in combination with a fuel container or tank 150, for example, in order to detect the presence and accumulation of contaminants within the fuel tank 150. In various embodiments, the impedance sensor arrays 102 can be placed at one or multiple locations within the fuel tank 150 and/or can be otherwise associated with the fuel tank 150 (e.g., positioned within in-line components coupled to the fuel tank 150). For example, in the embodiment shown in FIG. 1, the impedance sensor arrays 102 are placed at multiple locations along a bottom surface of the fuel tank 150. As another example, in the embodiment shown in FIG. 2, an impedance sensor array 102 can additionally be positioned within a conduit 152 coupled to the fuel tank 150. The microbial biosensor system 100 can further include a computer system 110 coupled to the impedance sensor arrays 102. The impedance sensor arrays 102 can be used to detect microbial growth within the fuel tank 150 because the growth of microbes (e.g., as biofilms) on or in the vicinity of the impedance sensor arrays 102, causes a corresponding change in impedance across the impedance sensor arrays 102. Accordingly, the computer system 110 can be configured to detect microbial growth within the fuel storage tank 150 based on changes in impedance across the impedance sensor arrays 102 (i.e., a change in the impedance signal output by the impedance sensor arrays 102) over time.

The sensor components of the impedance sensor arrays 102 can be small, compact, chip-based devices, which is described in further detail below. Many separate impedance sensor arrays 102 (also referred to as “modules”) can be incorporated into the fuel tank 150. Advantageously, this allows for multiple impedance sensor arrays 102 to be used to monitor for potential microbial growth within the fuel tank 150 in parallel with each other. In various embodiments, the impedance sensor arrays 102 can be stabilized to the floor (as shown in FIGS. 1 and 2) or walls of the tank 150 with magnets, held to the bottom of the tank 150 under their own weight, and so on. This network of impedance sensor arrays 102 enables the microbial contamination and activity to be mapped throughout the tank (including regions near shallow puddles of water on the floor on the fuel tank 150). This differentiates from other systems that monitor only at a single point (e.g., the access port or the ATG), which may not be near the initial source of contamination. In addition to enabling easy integration with the ATG, the compact design of the sensor modules enables them to be readily inserted in-line, e.g. either in series or in parallel with a flow meter. By being able to position multiple impedance sensor arrays 102 throughout the fuel tank 150 and the associated components, the microbial biosensor system 100 can generate a comprehensive map of microbial growth throughout the fuel tank 150 and detect microbial growth at an early stage (i.e., avoiding situations where microbial growth goes undetected for an extended period of time because the growth is originating at a location distal to the single monitored location in some conventional systems).

As noted above, each impedance sensor array 102 responds quantitatively to the amount of microbial activity within the sensor array's 102 immediate vicinity. Accordingly, in some embodiments, the computer system 110 can be configured to classify the response of each of the impedance sensor arrays 102 into one or more of multiple categories for easy interpretation by the tank operator, such as “Acceptable,” “Moderate/Warning,” and “Heavy/Action.” An “Acceptable” status could indicate that routine monitoring should be continued, that test frequency can be reduced if three successive results are determined to be acceptable, and/or that users should continue draining off any water found in the fuel tank 150 as part of ongoing standard maintenance. A “Moderate” status could indicate that users should increase the frequency at which water is being drained from the fuel tank 150 and the associated filter vessels, settling protocols should be improved prior to tank release, the fuel tank 150 should be retested within one month, and that the frequency of the routine monitoring should be increased if successive results are not acceptable (i.e., are “Moderate” or “Heavy”). A “Heavy” status could indicate that the fuel tank 150 should be immediately retested to confirm the “Heavy” status determined by the computer system 110, users should increase the frequency at which water is being drained from the fuel tank 150 and the associated filter vessels, settling protocols should be improved, bulk fuel samples from the affected fuel tank 150 should be tested to further determination the degree of contamination, and so on. These classifications can be output by the computer system 110 (e.g., via a display screen and an associated graphical user interface) to provide users with clear, unambiguous statuses or recommendations for the state of microbial growth within the fuel tank 150. In one embodiment, the computer system 110 can be configured to monitor the state of microbial growth within the fuel tank 150 by comparing the outputs (e.g., signals) from the impedance sensor arrays 102 to a baseline or threshold impedance output. For example, when the impedance detected by one or multiple of the impedance sensor arrays 102 exceeds a threshold level, the computer system 110 can determine that there is microbial growth within the vicinity of the impedance sensor array(s) 102. Further, the computer system 110 can be configured to quantify the amount of microbial growth based upon the difference between the measured impedance and the baseline or threshold impedance values. In other words, in some embodiments, as the measured impedance values at the impedance sensor arrays 102 increase, the computer system can quantify the amount of microbial growth within the vicinity of the impedance sensor arrays 102. Still further, the computer system 110 can be configured to compensate for whether the impedance sensor arrays 102 (or individual sensors thereof) are present within the fuel or aqueous phases within the fuel tank 150, as described in greater detail below. In one embodiment, the computer system 110 can use different baselines or thresholds for the different phases or can modify the baselines or thresholds according to the phase type in which the impedance sensor arrays 102 (or individual sensors thereof) are located. In some embodiments, the baseline or threshold impedance values can be predetermined or preprogrammed into the computer system 110. In other embodiments, the baseline or threshold impedance values can be characterized by the computer system 110 (e.g., the computer system 110 can determine the impedance or threshold impedance values upon activation or when initiated by a user). In some embodiments, the baseline or threshold impedance values could include an average impedance across the sensors 104 of an impedance sensor array 102 or an average impedance across the impedance sensor arrays 102 of the microbial biosensor system 100.

In one embodiment, the microbial biosensor system 100 can be a standalone system that is incorporated into the fuel tank 150. In another embodiment, such as is shown in FIG. 2, the microbial biosensor system 100 can be retrofitted into or otherwise incorporated with the ATG system 120 of the fuel tank 150. In particular, due to the small size of the sensors, the impedance sensor arrays 102 can be readily attached onto an existing ATG system 120. The sensor chips can be integrated into any number of housing components for protection and easy removal/replacement. For example, the impedance sensor arrays 102 could be integrated along the length of the ATG probe shaft or directly integrated onto fuel/water float sensor of the ATG. Notably, the impedance sensor arrays 102 can be placed in any orientation (i.e., vertical or upside down) within the liquid to prevent the settling of potentially interfering non-biological particulates onto the sensing surface of the impedance sensor arrays 102.

Embodiments where the microbial biosensor system 100 is incorporated into the ATG system 120 can be beneficial because the components of the ATG system 120 can act as a physical support onto which the impedance sensor arrays 102 or other components of the biosensor system 100 can be attached or anchored. Further, the sensor output from the microbial biosensor system 100 can be integrated into the ATG's graphical user interface 122 to enable easy and understandable readout by the tank operator. Further, the microbial biosensor system 100 is low-profile and has a compact form factor for easy integration onto ATG systems 120 without adding any significant size or weight.

As described in further detail below, the microbial biosensor system 100 can be used to measure microbial growth in heterogeneous, multiphase environments (e.g., within a fuel tank 150), unlike currently used methods. The microbial biosensor system 100 offers a continuous, automated, in-tank, and sensitive diagnostic platform for detecting microbial growth. Further, the presently described microbial biosensor system 100 offers several benefits over current monitoring systems, including not requiring any reagents or other consumables, not requiring any samples to be processed or any other operations to be performed manually by users, taking advantage of multiple parallel sensor arrays 102 to provide additional functionality (which is described below), providing immediate sensor readout, using compact electronic components, having low power requirements, being mechanically robust (i.e., using no moving parts), and using low cost, readily replaceable components. Further, in some embodiments, the microbial biosensor system 100 can be retrofitted or otherwise incorporated into an existing ATG system.

### Sensor Arrays

In one embodiment, the impedance sensor array 102 can include one or more microelectrodes (i.e., sensors) patterned on a metal thin-film pattern on a support material (e.g., a silicon wafer). The microelectrodes can be arranged in an interdigitated array, for example. In various embodiments, the impedance sensor array 102 can be provided in various numbers, arrangements, and sizes of sensors. For example, the impedance sensor array 102 could include a 5×5 mm² array of sensors spaced with 5 μm spacing therebetween. In other examples, the impedance sensor array 102 can include microscale sensors (e.g., sensors <<1 mm²). Microscale sensors can be advantageous because the small size allows many of such sensors to be fabricated in parallel on the same device. Further, smaller electrodes provide higher sensing sensitivity, enabling earlier detection of cells and biofilm formation. Placing many discrete microsensors in parallel allows for the presence of biomass to be monitored over a larger area without sacrificing the sensitivity of the individual microsensors. The result is a large-area, highly sensitive array of sensor “pixels,” each of which produce an individual response, but can be collectively used to improve diagnostic capability.

In some embodiments, the impedance sensor array 102 can also be fabricated in parallel with highly scalable batch production afforded by the semiconductor manufacturing processes. This aspect, coupled with the overall simplicity and few process steps, allows for low-cost devices to be produced. Despite having many microsensor components, an impedance sensor array 102 can be used as a disposable unit, preventing the need for cleaning or maintenance, and can simply be replaced at any time. The low cost also allows for multiple impedance sensor arrays 102 to be used simultaneously in a single fuel system to maximize diagnostic capability.

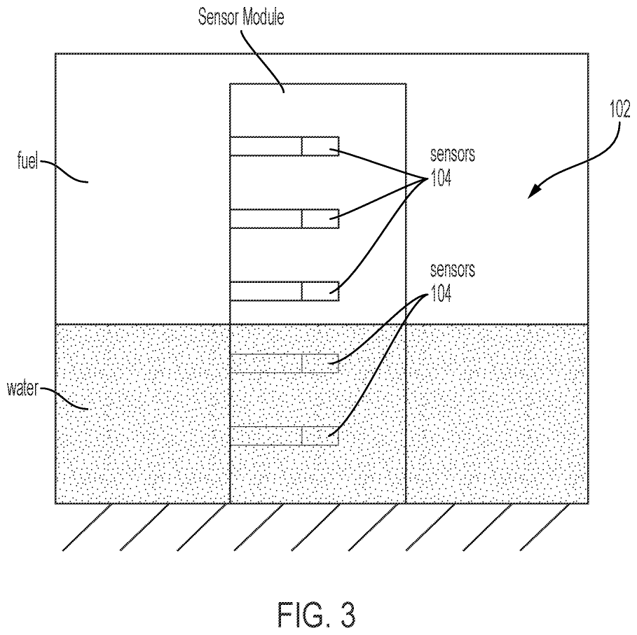

One embodiment of an impedance sensor array 102 is shown in FIG. 3. In this embodiment, the impedance sensor array 102 includes multiple individual impedance sensors 104 arranged in a vertical or stacked arrangement. As noted above, each of the impedance sensors 104 can be configured to measure the impedance thereacross or provide an impedance output signal, which can be received by the computer system 110 for detecting the presence or accumulation of microbial growth in the vicinity of the sensors 104.

### Multivariate/Multiplex Sensor Data

As discussed above, uses many individualized impedance sensors 104 and/or impedance sensor arrays 104 provides improved sensitivity in detecting microbial growth within a fuel tank 150 over conventional systems that use a single biosensor. However, each of these sensors 104 must be individually interrogated by the computer system 110 to monitor the microbial growth. Fortunately, rapid measurement and signal acquisition is an advantage of electronic sensors, where a measurement is typically performed in the millisecond time scale or faster. In one embodiment, the sensors 104 can be individually interrogated in a serial manner within a relatively short time frame (˜1 second). Serial operation also simplifies the control electronics.

The large numbers of parallel sensors 104 also enable implementation of novel data processing techniques to improve sensitivity, mitigate noise, and compensate for signal drift. As on illustrative example, in one embodiment an impedance sensor array 102 could comprise a 10×10 array of discrete interdigitated electrode impedance microsensors, each 50×50 μm². This yields an array area of 100 discrete sensors within a 1×1 mm² area. If a single bacteria cell becomes randomly immobilized on the surface of the sensor array, it slightly perturbs the impedance signal of the specific microsensor on which it is bound. As the cell begins to divide, and biological material accumulates onto the microsensor, the sensor response changes further. The resulting signal is compared to the background signal of the surrounding 99 cell-free sensors. This large number of “reference” microsensors provides a highly convenient and reliable baseline signal that corrects for issues such as signal drift, which depend upon inevitable changes in temperature, pH, concentration of dissolved components (organics, water, ions), etc. Given that all these microsensors of a single array are effectively within the same location, the local conditions of each sensor pixel are effectively identical and therefore provide a reliable baseline signal that can be subtracted from the signal that is observed to deviate from the average.

In various embodiments, the aforementioned steps or techniques executed by the computer system 110 can be embodied as hardware, software, firmware, or any combination thereof. For example, the steps described above can be embodied as computer executable instructions stored in a memory of the computer system 110 that, when executed by a processor, cause the computer system 110 to perform the steps.

### Decontamination Monitoring

In addition to being used to monitor or measure contamination over time, the microbial biosensor system 100 described herein could also be used to quantitatively monitor decontamination processes. When a fuel tank 150 is determined to be contaminated, a common treatment is to use a biocide and/or perform a recirculation of the fuel through a filtration system to remove the contamination. When the surfaces within the tank 150 are subsequently cleaned through this process, either through mechanical or chemical degradation of the biofilms, then the surfaces of the sensors can be cleaned in a similar manner. The removal of biomaterial from the sensors in turn results in a change of the impedance signal output by the impedance sensor array 102. This offers a potential to monitor the decontamination process in much the same way as the sensors are used to monitor the contamination. Afterwards, the resulting sensor signal can be used as the new baseline signal for subsequent monitoring of contamination over time.

### Water Level Monitoring

In addition to the aforementioned uses, the microbial biosensor system 100 described herein could also be used to measure the water level within the fuel tank 150. Due to its dielectric and conductive properties, the fuel phase demonstrates an impedance value that is much higher than the aqueous phase. Accordingly, the differences in the impedance values of the different phases provide a convenient “self-calibrating” capability whereby the location of a microsensor can be determined by simply observing the baseline impedance signal. The impedance signal of the aqueous phase and the interface changes by approximately 20-30% from initial no contamination to later stage heavy microbial contamination. However, the impedance signal of the sensors in the fuel demonstrated an impedance 2-3 orders magnitude higher than that of the aqueous and interface samples. The baseline impedance signal can act as an indicator of water level, which in turn enables the sensing algorithm to automatically switch between water or fuel phase analysis. As discussed above, the microelectrode impedance sensors can be very small (<1 mm²) and readily produced as an array of individual sensors on the same chip. One or more chips may be incorporated into a single sensor module deployed at the bottom of the tank. A “stack” of individual sensors can therefore measure the impedance response, both from the water/fuel phase, and the microbial growth, as shown in FIG. 3. These can then determine the level of the water with precision equivalent to the separation of the microsensors, which can be readily designed to be <1 mm. This also allows for monitoring of microbial contamination and water level for very shallow water bottom “puddles” that accumulate in the tank, but may not be measurable with a standard ATG or conventional sampling techniques.

### Examples

Experimental data demonstrating how the microbial biosensor system 100 can detect the presence of microorganisms commonly found in fuel storage containers can be found below in TABLE 2. In particular, TABLE 2 illustrates how some representative microorganisms can be detected by the microbial biosensor system 100, including Pseudomonas putida (a gram-negative bacterium), Gordonia (a gram-positive bacterium), Hormoconis resinae (“HR,” a filamentous fungus), and Yarrowia lipolytica (“YaLi,” a fungal yeast). In these particular implementations, a positive detection signal was defined to be 5-10% of the maximum impedance signal change observed over the course of the experiment. The microbial sensor system 100 was experimentally determined to be capable of detecting Gordonia, YaLi, and HR at equal to or less than 1000 CFU or cells per mL (as determined by the culture plating and qPCR). Furthermore, most samples demonstrate a measurable impedance signal well under the limit of detection target of 750 CFU/mL. TABLE 2 summarizes the culture plating and qPCR results that correspond to the point where the impedance signal is detected for the presence of microorganisms. Note that in TABLE 2, a “*” indicates that the measurement was difficult to determine due to the significant drop in 16S genes present at 100 hrs.

TABLE 2

| | | Sensor System vs. | | Sensor System vs. | |
|---|---|---|---|---|---|
| Organism | Phase | Culture Plating | | qPCR | |
| Pseudomonas | Fuel | — | | — | |
| putida | Interface | 2 × 10⁴ | CFU/mL | 1 × 10⁶ | cells/mL |
| | Aqueous | 2 × 10⁴ | CFU/mL | 1 × 10⁶ | cells/mL |
| Gordonia | Fuel | — | | — | |
| | Interface | 10 | CFU/mL | <1 | cells/mL |
| | Aqueous | 10 | CFU/mL | <1 | cells/mL |
| Yarrowia | Fuel | — | | — | |
| lipolytica | Interface | 100 | CFU/mL | 100 | cells/mL |
| | Aqueous | 100 | CFU/mL | 100 | cells/mL |
| Hormoconis | Fuel | — | | — | |
| resinae | Interface | <1 | CFU/mL | <1 | cells/mL |
| | Aqueous | <1 | CFU/mL | <1 | cells/mL |

| Mixture | Fuel | — | | — |
|---|---|---|---|---|
| | Interface | 2 × 10⁷ | CFU/mL | * |
| | Aqueous | 1 × 10⁴ | CFU/mL | * |

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A system for detecting microbial contamination in a liquid storage container, the system comprising:
an impedance sensor array positioned within the liquid storage container, the impedance sensor array configured to measure an impedance across the impedance sensor array; and
a computer system coupled to the impedance sensor array, the computer system configured to:
receive the impedance from the impedance sensor array,
compare the impedance to a threshold impedance, and
determine whether microbial growth is present within a vicinity of the impedance sensor array based on the comparison.

2. The system of claim 1, wherein the computer system is used to further quantify an amount of the microbial growth based on the comparison.

3. The system of claim 1, wherein the computer system is further configured to output a user alert based on the comparison.

4. The system of claim 1, wherein the threshold impedance has been previously characterized for the liquid tank by the computer system.
